(19) United States
(12) Patent Application Publication
(10) Pub. No.: US 2010/0160978 A1
(43) Pub. Date: Jun. 24, 2010

(54) BONE SCREW ASSEMBLY WITH NON-UNIFORM MATERIAL

(76) Inventor: John Carbone, Lutherville, MD (US)

Correspondence Address: CARTER, DELUCA, FARRELL & SCHMIDT, LLP, 445 BROAD HOLLOW ROAD, SUITE 420, MELVILLE, NY, 11747, US

(21) Appl. No.: 12/645,011

(22) Filed: Dec. 22, 2009

Related U.S. Application Data
(60) Provisional application No. 61/203,481, filed on Dec. 23, 2008.
(60) Provisional application No. 61/203,502, filed on Dec. 23, 2008.

(51) Int. Cl. A61B 17/86

(52) U.S. Cl. 606/305

(57) ABSTRACT

A bone anchor comprises a screw having a threaded shank portion and a head portion, and a housing assembly. At least the threaded shank portion is formed from a titanium or titanium alloy and at least a portion of the housing is formed from a stronger material such as cobalt chrome. The housing assembly has a passageway extending therethrough and a proximal end and a distal end. A saddle is defined within the proximal end of the housing assembly and is configured to retain a portion of a rod therein. The distal end of the housing assembly is configured for securely engaging the head portion of the screw such that the screw is at least one of rotatable and pivotable with respect to the housing.

### CROSS-REFERENCE TO RELATED APPLICATIONS

This application claims the benefit of and priority to U.S. Provisional Application Nos. 61/203,481 and 61/203,502, both of which were filed on Dec. 23, 2008, and are hereby incorporated by reference herein.

### BACKGROUND

1. Technical Field

The present disclosure relates to bone anchors and, more particularly, to pedicle screw assemblies made from non-uniform materials.

2. Background of Related Art

It is a common surgical procedure to stabilize and fix bones and bone fragments in a particular spatial relationship with fixation devices to correct the location of skeletal components due to injury or disease. This can be accomplished by using a number of fixation devices such as bone pins, anchors, or screws placed in bone across a discontinuity (e.g., a fracture) in the bone, bone fragments, adjacent vertebrae, or joints. These fixation devices can be connected by a rod to maintain a desired spatial relationship. In some cases, these fixation devices may be permanently implanted. In other cases, these fixation devices may be implanted only as a temporary means of stabilizing or fixing the bones or bone fragments. It is also common that fixation devices that are intended to be permanently implanted require subsequent modifications as the dynamics of a patient's condition warrant.

Spinal fixation apparatuses are widely employed in surgical procedures for correcting spinal injuries and diseases. A common desire for spine surgery, especially for scoliosis surgeries, is the need for a stronger, stiffer rod, typically made of Cobalt Chrome (CoCr). These rods provide the needed strength to correct the deformity, but due to the strength of typical spine screws, the rod may dislocate from the spinal fixation device under bodily forces experienced after implantation. Such dislocation can be caused either by axial slip, i.e., sliding of the rod end through the spinal fixation device along the axis of the rod, or radial displacement of the rod out of the screw. Either type of dislocation can happen with any type of spinal fixation device, including both taper lock style screws and set screw style screws.

To prevent these potential problems, a stronger housing that could withstand the increased force required to lock and unlock the spinal rod is needed. However, while the housing is desirably made of a stronger and stiffer material, the screw itself may need to be formed of a less rigid, bone interface material. Specifically, a screw made of a titanium alloy such as Ti-6Al-4V has been shown to be very compatible as a bone interface material.

### SUMMARY

In accordance with the present disclosure, a bone anchor is provided. The bone anchor includes a screw and a housing assembly. The screw has a threaded shank portion and a head portion. The housing assembly has a passageway extending therethrough. A saddle is defined within the proximal end of the housing assembly and is configured to retain a portion of a rod therein. The distal end of the housing assembly is configured to securely engage the head portion of the screw such that the screw is rotatable and/or pivotable with respect to the housing. At least a portion of the screw and at least a portion of the housing assembly are made of different material.

In one embodiment, the entire housing assembly is made from cobalt chrome. In another embodiment, only a portion of the housing assembly is made from cobalt chrome. The entire screw may be made from titanium or another biocompatible material.

In another embodiment, the housing assembly includes a housing part, a coupling, an insert, and a set screw. The housing part includes two proximally extending fingers defining the saddle therebetween. The coupling is disposable within the housing part and is configured to engage the head portion of the screw. The insert is configured to engage the housing part to secure the coupling and the head portion of the screw therebetween. The set screw is configured to engage the housing part and to secure the screw in position.

In yet another embodiment, the housing assembly includes a collet, a coupling, and a pin. The collet has an opening extending therethrough and includes two proximally extending fingers defining the saddle therebetween. The collet is configured to accept, through its distal end, the head portion of the screw. The head portion of the screw is partially insertable into the opening in the collet. The coupling also has an opening extending therethrough. The coupling is configured to pass proximally over the shank portion of the screw to ultimately surround the collet, thereby securing the head portion of the screw between the collet and the coupling. The pin is configured to engage the collet with the coupling to thereby secure the collet and the coupling to each other.

### BRIEF DESCRIPTION OF THE DRAWINGS

Embodiments of the presently disclosed bone screw assembly are described herein with reference to the accompanying drawings, wherein:

FIG. 1 is an exploded perspective view of a bone anchor assembly according to the present disclosure;

FIG. 2 is a side view of the bone anchor of FIG. 1;

FIG. 3 is a side, cross-sectional view of the bone anchor of FIG. 2, taken across section line 3-3;

FIG. 4 is an exploded perspective view of a bone anchor assembly in accordance with another embodiment of the present disclosure;

FIG. 5 is a side view of the bone anchor of FIG. 4; and

FIG. 6 is a side, cross-sectional view of the bone anchor of FIG. 2 taken across section line 6-6.

### DETAILED DESCRIPTION OF THE EMBODIMENTS

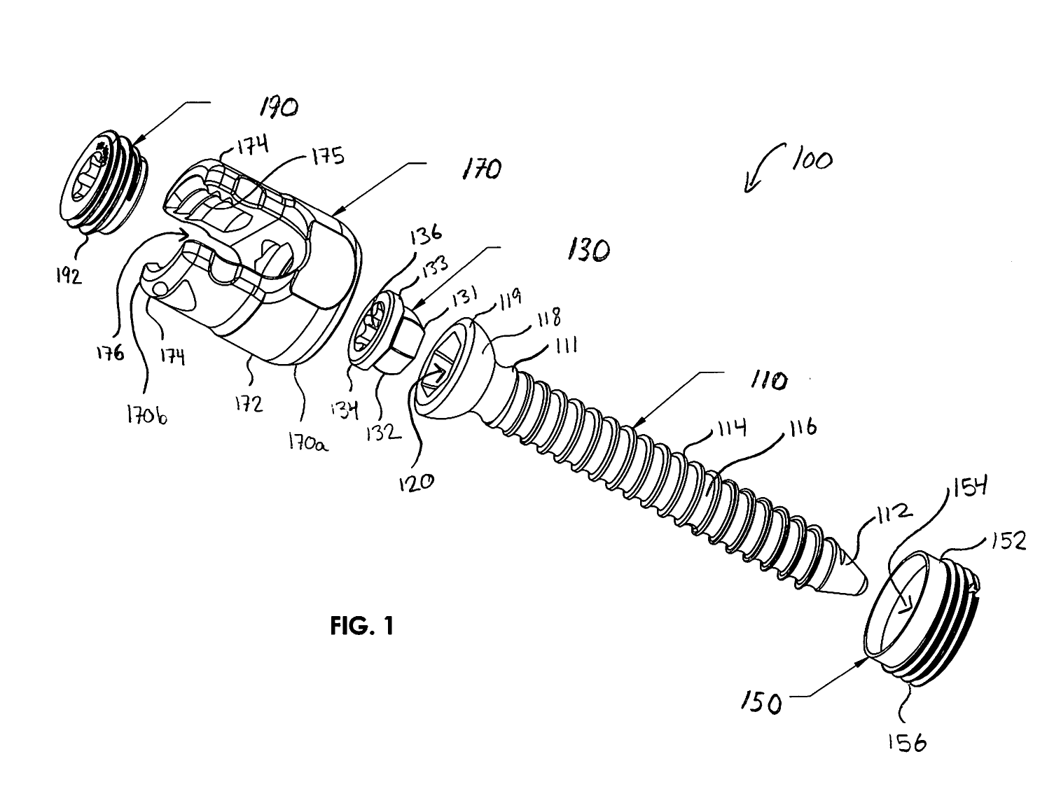

Turning to FIG. 1, a bone anchor 100 is shown including a pedicle screw 110, a coupling 130, an insert 150, a housing 170, and a locking element 190. During assembly of the bone anchor 100, the coupling 130 is positioned at a proximal end 111 of the pedicle screw 110 and the housing 170 is positioned over the coupling 130 and pedicle screw 110. The insert 150 is then passed over the distal end 112 of the pedicle screw 110 and is translated distally along the shaft 116 of the screw 110 towards the housing 170. The insert 150 is then engaged with a distal end 170a of the housing 170 to hold the bone anchor 100 together. The locking element 190 may then be engaged with a proximal portion 170b of the housing 170 to lock the screw 110 in place. Alternatively, the screw may be a top loading screw, such that an insert would not be required. In such an embodiment, the screw would first be inserted through the housing. The housing would be configured such that the head of the screw is inhibited from passing through the distal end of the housing. As mentioned above, the locking element may then be engaged with the housing to lock the screw in place. The specific arrangements and interconnections of the various components of bone anchor 100 will be described in further detail hereinafter.

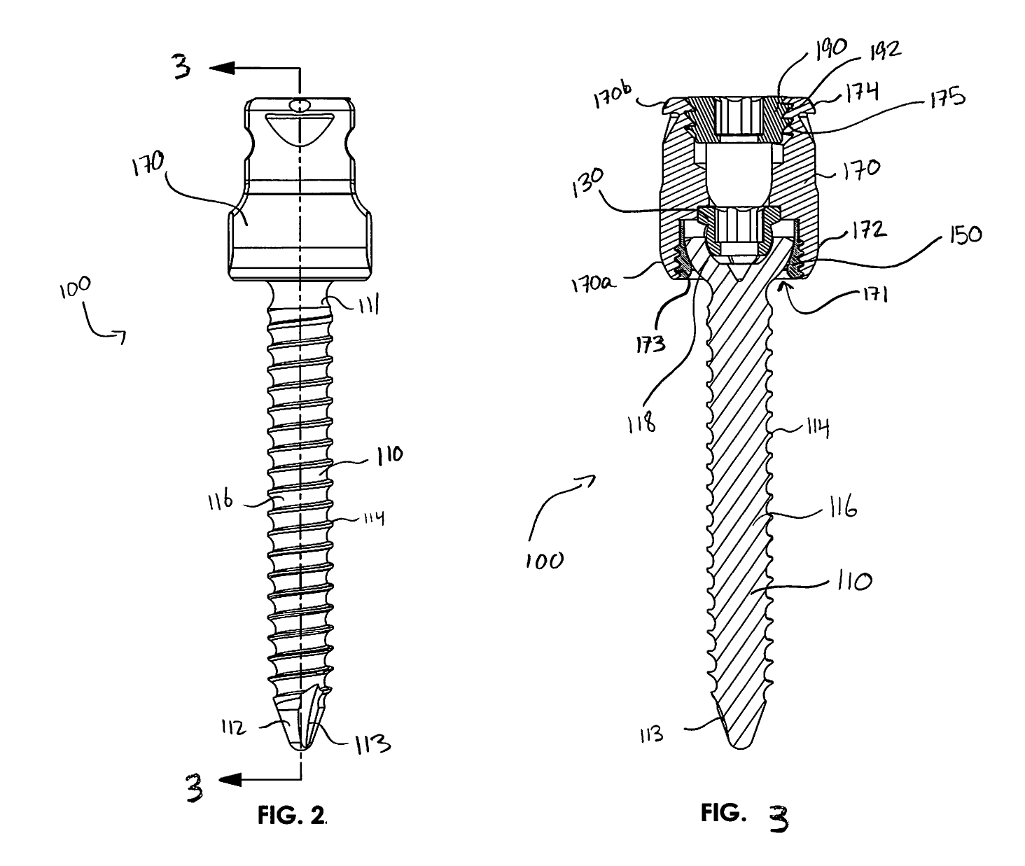

With reference now to FIGS. 1-3, the distal end 170a of housing 170 includes an annular body portion 172 having an opening 171 therethrough. The proximal end 170b of housing 170 has a pair of upstanding fingers 174, defining a U-shaped saddle therebetween. The saddle 176 is configured for receiving a portion of a rod member (not shown). The opening 171 defined at distal end 170a of body portion 172 includes a generally helical thread 173 on the inner surface of body 172. Thread 173 is adapted to threadingly engage a corresponding thread 156 of the insert 150, as will be described in greater detail below. Similarly, each of the fingers 174 extending from housing 170 includes a portion of a generally helical thread 175 formed on the inner surface of the fingers 174. The thread 175 is configured for threadably engaging a corresponding thread 192 on locking element 190. As shown in FIG. 1, the locking element 190 may be a set screw 190. Alternatively, the locking element 190 may be any other threaded component or wedge component (see, for example, U.S. Pat. Nos. 6,090,111 to Nichols and 7,608,095 to Yuan, et al.) as known in the art. The opening 171 at the distal end 170a of housing 170 is capable of partially receiving the coupling 130 and the pedicle screw 110, while not allowing the coupling 130 or the pedicle screw 110 to pass completely therethrough. In other words, the distal portion 170a of housing 170 is configured to retain the coupling 130 and head portion 118 of screw 110 therein.

As shown in FIG. 1, the coupling 130 has a generally annular body 132 at a distal end 131 thereof and an annular flange 134 at a proximal end 133 thereof. The annular body 132 is shown having a hexagonally-shaped outer surface that tapers distally from the annular flange 134 to the distal end 131 of the coupling 130. However, it is also contemplated that the annular body 132 be configured in different arrangements. For example, a plurality of outwardly extending knobs (not shown) may be provided extending from the annular body 132. The annular flange 134, disposed at the proximal end 133 of the coupling 130, has an outer diameter that is greater than the outer diameter of the body portion 132. Additionally, a recess 136 is formed in the proximal end of the coupling 130. The recess 136 is configured and adapted for releasable engagement with a driving tool (not shown), as is known in the art. Although the recess 136 is illustrated with a six-pointed star pattern, other suitable configurations corresponding to the driving tool to be used are also contemplated.

Continuing with reference to FIGS. 1-3, the pedicle screw 110 will be discussed in detail. The pedicle screw 110 includes a shank 116 having a helical thread 114 formed thereon. A cutting portion 113 is formed at a distal end 112 of the pedicle screw 110. A head portion 118 is located at a proximal end 111 of the pedicle screw 110. Head portion 118 is generally hemispherical in shape wherein the flat end of the hemisphere forms the proximal end 119 of the head portion 118. A recess 120 is defined within the flat, proximal end 119 of the head portion 118. The inner surface of the recess 120 tapers inwardly and distally and is hexagonal in shape, such that the outer surface of the body 132 of the coupling 130 and the inner surface of recess 120 of the head portion 118 are complementary to one another. As can be appreciated, both the inner surface of the head portion 118 of the screw 110 and the outer surface of the body portion 132 of the coupling 130 may define different shapes or arrangements, as long as they are complementary to one another. For example, in the configuration wherein the annular body 132 includes a plurality of outwardly extending knobs (not shown), the head portion 118 would include a complementary configuration, e.g., a plurality of segments having gaps therebetween such that each gap is adapted to releasably receive a knob therein. Due to the complementary-shaped configuration, when the coupling 130 is engaged within the recess 120 of the head portion 118 of screw 110, the coupling 130 and the pedicle screw 110 are rotatably coupled to one another such that rotation of the coupling 130 causes a corresponding rotation of the pedicle screw 110.

The insert 150, as shown in FIG. 1, is an annular ring 152 having an opening 154 extending therethrough. The opening 154 has a diameter that is greater than the shank 116 of the pedicle screw 110 but smaller than the head portion 118 of the pedicle screw 110. A generally helical thread 156 is disposed on the outer surface of the annular ring 152. The threads 156 are configured to mate with the threads 173 defined on the inner proximal surface of the housing 170 (FIG. 3).

Referring again to FIGS. 1-3, assembly and usage of the screw assembly 100 will now be discussed in detail. Initially, the coupling 130 is seated within the recess 120 defined at the proximal end 119 of head portion 118 of pedicle screw 110, such that the coupling 130 and head portion 118 are engaged via their respective complementary-shaped hexagonal surfaces. As a result, the coupling 130 is slidably received in the recess 120. The tapered outer surface of body portion 132 mates with the tapered inner surface of the recess 120 and allows the coupling 130 to be seated within the recess 120.

As previously discussed, when the coupling 130 is seated in the recess 120 of the pedicle screw 110, rotation of the coupling 130 causes a corresponding rotation of the pedicle screw 110, thereby allowing the pedicle screw 110 to be inserted and removed from a target location. The interaction of coupling 130 and recess 120 in screw 110 permits the screw 110 to be driven in response to a driver tool (not shown) which engages the coupling 130 even if the screw 110 is disposed at an angle relative to the coupling 130. Thus, the screw shaft 116 and driving tool (not shown) can be out of alignment during insertion of the screw 110 into bone.

During assembly, the coupling 130 and the pedicle screw 110 are inserted into the housing 170. The distal opening 171 in the housing 170 has a greater diameter than the outer diameters of either the head 118 or the coupling 130. The insert 150 is then slid over the shank 116 of the pedicle screw 110 and threaded, or wedged, onto the distal end 170a of the housing 170. The opening 154 of the insert 150 has a diameter that is less than that of the head 118 of the pedicle screw 110, thereby inhibiting the pedicle screw 110 from passing through the opening 154 of the insert 150. By threading the insert 150 onto the distal end 170a of the housing 170, the pedicle screw 110 and the coupling 130 are retained in the housing 170 and thereby form the assembled bone anchor 100. The pedicle screw 110 is rotatable and pivotable in relation to the housing 170.

After the bone anchor 100 is positioned at a desired location in a patient, a rod member (not shown) is placed in the saddle 176 and is retained within the housing 170 using a locking, or set screw 190. As the set screw 190 is tightened against the rod member (not shown), the rod member presses against the coupling 130, thereby pressing the head 118 of the pedicle screw 10 against the inner surfaces of the insert 150 and securing the pedicle screw 110 in position (i.e. locking the screw in place).

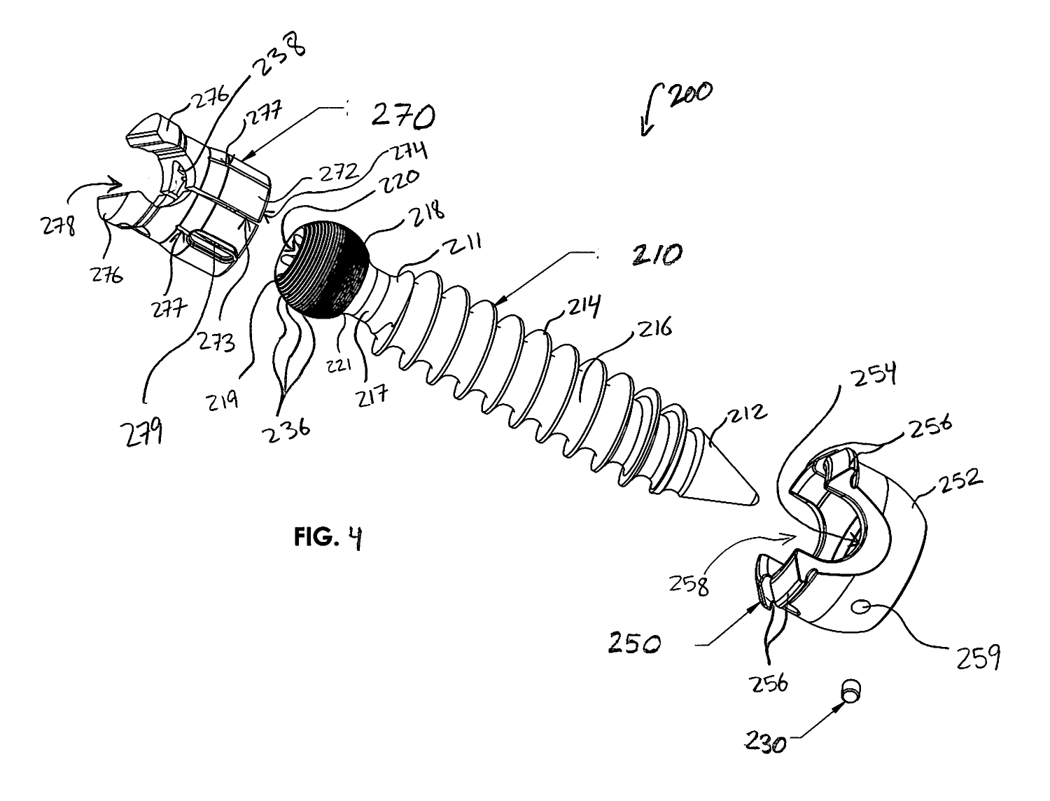

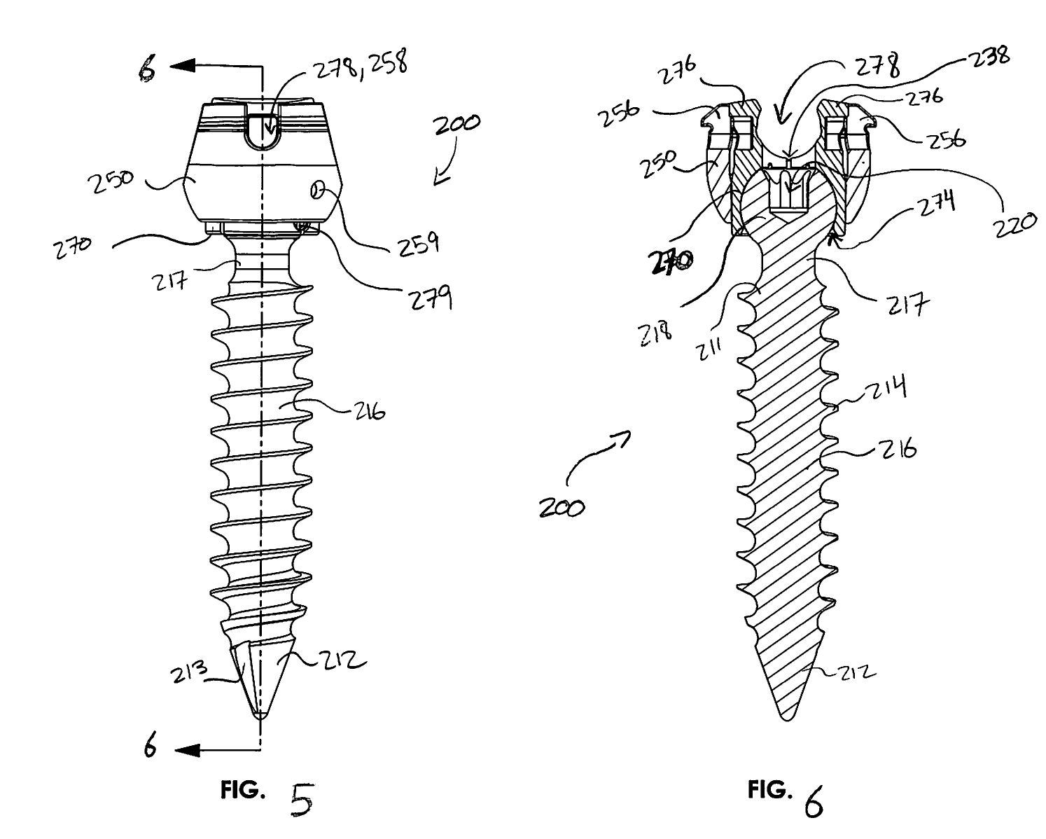

In accordance with another embodiment of the present disclosure, as shown in FIGS. 4-6, a bone anchor 200 is provided. The bone anchor 200 includes a pedicle screw 210, a pin 230, an outer collet 250, and an inner collet 270. The outer collet 250 includes an annular body portion 252 having an opening 254 extending axially therethrough. Additionally, the outer collet 250 includes a plurality of fingers 256 that extend proximally from the outer collet 250 and define a saddle 258 having a generally U-shaped configuration. The U-shaped saddle 258 is configured and dimensioned for receiving a portion of a rod member (not shown), similar to saddle 176 of bone anchor 100.

As shown in FIG. 4, the inner collet 270 has a generally cylindrical body portion 272 with an opening 274 extending axially therethrough. A pair of upstanding wings 276 defines a saddle 278 having a generally U-shaped configuration. The saddle 278 of inner collet 270, along with saddle 258 of outer collet 250, is configured and dimensioned for receiving a rod member (not shown). The body portion 272 includes a slot 273 that extends from the nadir of the saddle 278 towards the distal end of the body portion 272 and essentially bisects the body portion 272 along a central axis, thereby defining left and right sections of the body portion 272. Preferably, the slot 273 does not extend all the way through the body portion 272. This arrangement permits each of the wings 276 to flex towards and away from each other, thereby varying the dimensions of the saddle 278 according to the flexure of the wings 276. As the wings 276 are moved closer to each other, the saddle 278 decreases in size. On the other hand, when the wings 276 are moved away from each other, the saddle 278 increases in size. Allowing the saddle 278 to vary in size permits the inner collet 270 to accommodate rods (not shown) having differing diameters. Additionally, the compression of the wings 276 towards each other increasingly engages the outer surface of a rod located in the saddle 278, thereby frictionally securing the rod in a desired position.

In addition, the body portion 272 of inner collet 270 may include a plurality of grooves (not explicitly shown) that extend to the distal end of the body portion 272 and which are open at the distal end of the body portion 272. The grooves extend vertically into each of the wings 276, and define front and rear portions of the body portion 272. As configured, the grooves permit the front and rear sections of the body portion 272 to flex relative to one another along the axis defined by the slot 273. The body portion 272 also includes a plurality of notches 277 that are open at the distal end of the body portion 272 and extend towards the wings 276. The notches 277, in combination with the slot 273 and the grooves (not shown), allow arcuate sections of the body portion 272 to flex inwardly and outwardly in response to compressive and tensile forces applied to the inner collet 270.

With continued reference to FIG. 4, the pedicle screw 210 includes a shank portion 216 having a helical thread 214 formed thereon. A cutting portion 213 (FIG. 5) is formed at a distal end 212 of the pedicle screw 210. A generally spherical head portion 218 is disposed at a proximal end 211 of the pedicle screw 210. The head portion 218 includes a plurality of grooves 236 formed thereon and has an outer diameter that is greater than the outer diameter of the shank 216. On the proximal end 219 of the head 218, a recess 220 is formed. The recess 220 is shown defining a six-pointed star configuration for receiving the operative end of a suitable driving tool (not shown), but it is contemplated that other configurations may be used. A neck 217 extends between a distal end 221 of the head portion 218 and the helical thread 214 at the proximal end 211 of the shaft 216. As configured, the neck 217 is unthreaded. As shown, at least a portion of the diameter of the neck 217 is less than the diameter of the head 218 and the major diameter of the threaded portion 214 of the shank 216.

Referring now to FIGS. 4-6, the pedicle screw assembly 200 will now be described as assembled for use. The inner collet 270 is seated atop the head 218 of pedicle screw 210. The opening 274 at the distal end of the inner collet 270 is dimensioned and configured for receiving the head portion 218 of screw 210, as discussed above. As such, the inner collet 270 and the head 218 are rotatable and pivotable in relation to each other, thereby allowing the pedicle screw 210 to be repositioned in a plurality of orientations relative to the inner collet 270. Next, the combination of the inner collet 270 and pedicle screw 210 is inserted into the outer collet 250, which is passed over the distal end 212 of the shaft 216 and moved proximally along the shaft 216 to engage the inner collet 270. The pin 230 is inserted through aperture 259 of the outer collet 250 and through slot 279 of inner collet 270 to align the inner collet 270 and the outer collet 250 for maintaining a fixed relationship therebetween. As assembled, the pedicle screw 210 is rotatable and pivotable in relation to the inner collet 270 and the outer collet 250, which are fixed relative to one another by pin 230. As mentioned above in connection with the previous embodiment, the rotatable and pivotable relationship between the outer collet 250 and the screw 210 allows the screw 210 to be driven in response to a driver tool (not shown) which engages the outer collet 250 (via recess 238) even if the screw 210 is disposed at an angle relative to the outer collet 250.

The bone anchors 100, 200, described above, may be composed of a range of materials. Biocompatible materials include, but are not limited to, titanium, titanium alloys, stainless steel, cobalt chrome and cobalt chrome alloys, ultra high molecular weight polyethylene, PEEK (polyetheretherketone), and other polymers such as polycarbonate urethane may be used. In one particular application, spinal surgery, strong, stiff rods, e.g., Cobalt Chrome (CoCr) rods, are used to help correct the spinal deformity. However, due to the strength of these rods as well as the spine screws used, the housing portion of the screw assembly may splay open or allow the rod to slip or turn in the saddle.

In accordance with the present disclosure, the housing, or saddle portion, is formed from a strong material that provides a greater holding force on the rod. CoCr is a preferred material for forming the housing portion due to its strength and stiffness. However, while Cobalt Chrome (CoCr) is a preferred material for forming housing portion, it is not necessarily a preferred material for forming the shank portion of the screw because CoCr may be too rigid to be used as a bone interface material. Instead, the preferred material for forming the screw shank is titanium, due to its lower modulus of elasticity and biocompatibility properties.

Put generally, due to the different interactions between and forces acting on the different parts of the screw assembly, it is preferable to construct the screw shank and the housing from different materials, according to the requirements for those specific parts. Furthermore, the sub-components of the housing, e.g., the coupling, insert, and/or collet, need not be made from the same material. For example, while it is preferred that the housing and set screw be made from CoCr, the other sub-components of the housing assembly may be made from titanium, depending on the intended usage of the bone anchor.

More specifically, bone anchor 100 is formed from titanium or a titanium alloy while at least the housing part 170 is made of CoCr. The housing part 170 is made from CoCr, which reduces splay and helps prevent the rod from slipping within the saddle 178 due to the strength and stiffness of CoCr. Further, the strength of CoCr allows housing part 170 to have a reduced thickness, or volume, while maintaining the structural integrity of the housing part 170. The screw 110, on the other hand, is constructed from titanium, which facilitates insertion and retention within bone due to its elasticity and biocompatible properties. The coupling 130, the insert 150, and the set screw 190 may each be made from one of CoCr, titanium or, titanium alloy. In one embodiment of bone anchor 200, screw 210 is made from titanium or titanium alloy, while the inner collet 270 and the outer collet 250 are made from either cobalt chrome (CoCr), titanium, or titanium alloy. Other materials are also contemplated for forming the components of bone anchors 100, 200, such that different materials may be used to form any or all of the components based upon the desired characteristics, e.g., strength or elasticity, of the specific component.

It will be understood that various modifications may be made to the embodiments of the presently disclosed pedicle screw construct. Therefore, the above description should not be construed as limiting, but merely as exemplifications of embodiments. Those skilled in the art will envision other modifications within the scope and spirit of the present disclosure.

What is claimed is:

1. A bone anchor comprising:
a screw having a threaded shank portion and a head portion;
a housing assembly having a passageway extending therethrough and a proximal end and a distal end, a saddle defined within the proximal end of the housing assembly and configured to retain a portion of a rod therein, the distal end of the housing assembly configured to securely engage the head portion of the screw such that the screw is at least one of rotatable and pivotable with respect to the housing; and
wherein at least a portion of the screw is formed from a first material and at least a portion of the housing assembly is formed from a second material that is stronger than the first material.

2. The bone anchor according to claim 1, wherein the housing assembly is made from cobalt chrome.

3. The bone anchor according to claim 1, wherein the screw is made from titanium.

4. The bone anchor according to claim 1, wherein the housing assembly includes:
a housing part including two proximally extending fingers defining the saddle therebetween;
a coupling disposable within the housing part and configured to engage the head portion of the screw;
an insert configured to engage the housing part thereby securing the coupling and the head portion of the screw therebetween; and
a set screw configured to engage the housing part to secure the screw in position.

5. The bone anchor according to claim 4, wherein the housing part is made from cobalt chrome.

6. The bone anchor according to claim 5, wherein at least one of the coupling, the insert, and the set screw are made from a different material.

7. The bone anchor according to claim 1, wherein the housing assembly includes:
an inner collet having an opening extending therethrough and including two proximally extending fingers defining the saddle therebetween, the inner collet configured to accept the head portion of the screw from a distal end of the inner collet and partially into the opening in the inner collet;
an outer collet having an opening extending therethrough; the outer collet configured to pass proximally over the shank portion of the screw to surround the inner collet, thereby securing the head portion of the screw therebetween; and
a pin configured to engage the inner collet with the outer collet to secure the inner collet and the outer collet to each other.

8. The bone anchor according to claim 1, wherein housing formed of the second material has a reduced volume in comparison to a housing formed of the first material.

9. The bone anchor according to claim 4, wherein the housing part and the set screw are formed from cobalt chrome.
